SEQUENCE LISTING (14 sequences)

SEQ ID NO: 1 - protein, 483 aa, Bacillus licheniformis [sequence omitted]

SEQ ID NO: 2 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 3 - protein, 584 aa, Bacillus species
ANTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 4 - protein, 584 aa, Bacillus flavothermus
VPVNGTMMQYFEWYLPDDGTLWTKVANNAQSLANLGITALWLPPAYKGTSSSDVGYGVYDLYDLGEFNQKGTVRTKYGTKTQYIQAIQAAHTAGMQVYADVVFNHKAGADGTELVDAVEVNPSDRNQEISGTYQIQAWTKFDFPGRGNTYSSFKWRWYHFDGTDWDESRKLNRIYKFRGTGKAWDWEVDTENGNYDYLMYADLDMDHPEVVSELKNWGKWYVTTTNIDGFRLDAVKHIKYSFFPDWLSYVRTQTQKPLFAVGEFWSYDISKLHNYITKTNGSMSLFDAPLHNNFYIASKSGGYFDMRTLLNNTLMKDQPTLAVTLVDNHDTEPGQSLQSWVEPWFKPLAYAFILTRQEGYPCVFYGDYYGIPKYNIPALKSKLDPLLIARRDYAYGTQHDYIDSADIIGWTREGVAEKANSGLAALITDGPGGSKWMYVGKQHAGKTFYDLTGNRSDTVTINADGWGEFKVNGGSVSIWVPKISTTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVP

SEQ ID NO: 5 - protein, 586 aa, Bacillus species
GSVPVNGTMMQYFEWYLPDDGTLWTKVANNAQSLANLGITALWLPPAYKGTSSSDVGYGVYDLYDLGEFNQKGTVRTKYGTKTQYIQAIQAAHTAGMQVYADVVFNHKAGADGTELVDAVEVNPSDRNQEISGTYQIQAWTKFDFPGRGNTYSSFKWRWYHFDGTDWDESRKLNRIYKFRGTGKAWDWEVDTENGNYDYLMYADLDMDHPEVVSELKNWGKWYVITTNIDGFRLDAVKHIKYSFFPDWLSYLRTQTQKPLFAVGEFWSYDINKLHNYITKTNGSMSLFDAPLHNNFYIASKSGGYFDMRTLLNNTLMKEQPTLSVTLVDNHDTEPGQSLQSWVEPWFKPLAYAFILTRQEGYPCVFYGDYYGIPKYNIPALKSKLDPLLIARRDYAYGTQHDYIDNADIIGWTREGVAEKANSGLAALITDGPGGSKWMYVGKQHAGKTFYDLTGNRSDTVTINADGWGEFKVNGGSVSIWVPKTSTTSQITFTVNNATTVWGQNVYVVGNISQLGNWDPVNAVQMTPSSYPTWVVTVPLPQSQNIQFKFIKKDGSGNVIWENISNRTYTVPTAASGAYTANWNVP

SEQ ID NO: 6 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 7 - protein, 485 aa, Bacillus species
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 8 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 9 - protein, 485 aa, Bacillus species
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 10 - protein, 485 aa, Bacillus species
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 11 - protein, 485 aa, Bacillus species
HHNGTNGTMMQYYEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWANFSVNGGSVSIWVNK

SEQ ID NO: 12 - protein, 480 aa, Bacillus species
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 13 - protein, 483 aa, Bacillus licheniformis
ANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEYLIKAWTHFHFPGRGSTYSDFKWYWYHFDGTDWDESRKLNRIYKFQGKYWDWEVSNENGNYDYLMYADIDYDHPDVVAEIKRWGTWYANELQLDGNRLDAVKHIKFSFLRDWVNHVREKTGKEMFTVAEYWQNDLGALENYLNKTNFNHSVFDVPLHYQFYAASTQGGGYDMRKLLNDTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWYDITGNRSEPVVINSEGWGEFHVNDGSVSIYVQR

SEQ ID NO: 14 - protein, 481 aa, Artificial Sequence
  Synthetic construct
VNGTLMQYFEWYTPNDGQHWKRLQNDAEHLSDIGITAVWIPPAYKAISQADVGYGAYDLYDLGEFHQKGTVRTKYGTKGELQSAIKSLHSRDINVYGDVVINHKAGADATEDVTAVEVDPADRNRVISGEHLIKAWTHFHFPGRGSTYSDFKWYWYHFDGTDWDESRKLNRIYKFQGKTWDWEVSNEFGNYDYLMYADFDYDHPDVVAEIKRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

(19) United States
(12) Patent Application Publication
(10) Pub. No.: US 2016/0122442 A1
(43) Pub. Date: May 5, 2016

(54) Process for Hydrolysis of Starch

(71) Applicant: Novozymes A/S, Bagsvaerd (DK)

(72) Inventors: Carsten Andersen, Vaerloese (DK); Paria Saunders, Knightdale, NC (US); Signe Eskildsen Larsen, Lyngby (DK); Scott R. McLaughlin, Wake Forest, NC (US); Chee Leong Soong, Raleigh, NC (US)

(73) Assignee: Novozymes A/S, Bagsvaerd (DK)

(21) Appl. No.: 14/782,640

(22) PCT Filed: Apr. 10, 2014
(86) PCT No.: PCT/US2014/033620

§ 371 Date: Oct. 6, 2015

Related U.S. Application Data
(60) Provisional application No. 61/810,396, filed on Apr. 10, 2013.
(60) Provisional application No. 61/818,120, filed on May 1, 2013.

(51) Int. Cl. C08B 30/12; C12N 9/26; C13K 7/00; C13K 11/00

(52) U.S. Cl. CPC C08B 30/12; C13K 11/00; C12N 9/2414; C13K 7/00; C12Y 302/01001

(57) ABSTRACT

The present invention relates to a process for enzymatic hydrolysis of granular starch into a soluble starch hydrolysate at a temperature below or just above the initial gelatinization temperature of said granular starch.

### REFERENCE TO A SEQUENCE LISTING

This application contains a Sequence Listing in computer readable form. The computer readable form is incorporated herein by reference.

### FIELD OF THE INVENTION

The present invention relates to a one step process for hydrolysis of granular starch into a soluble starch hydrolysate at a temperature below or just above the initial gelatinization temperature of said granular starch.

### BACKGROUND OF THE INVENTION

A large number of processes have been described for converting starch to starch hydrolysates, such as maltose, glucose or specialty syrups, either for use as sweeteners or as precursors for other saccharides such as fructose. Glucose may also be fermented to ethanol or other fermentation products such as lactic acid, citric acid, or crystalline sugars such as crystalline dextrose.

Starch is a high molecular-weight polymer consisting of chains of glucose units. It usually consists of about 80% amylopectin and 20% amylose. Amylopectin is a branched polysaccharide in which linear chains of alpha-1,4 D-glucose residues are joined by alpha-1,6 glucosidic linkages.

Amylose is a linear polysaccharide built up of D-glucopyranose units linked together by alpha-1,4 glucosidic linkages. In the case of converting starch into a soluble starch hydrolysate, the starch is depolymerized. The conventional depolymerization process consists of a gelatinization step and two consecutive process steps, namely a liquefaction process and a saccharification process.

Granular starch consists of microscopic granules, which are insoluble in water at room temperature. When an aqueous starch slurry is heated, the granules swell and eventually burst, dispersing the starch molecules into the solution. During this “gelatinization” process there is a dramatic increase in viscosity. As the solids level is 30-40% in a typical industrial process, the starch has to be thinned or “liquefied” so that it can be handled. This reduction in viscosity is today mostly obtained by enzymatic degradation. During the liquefaction step, the long-chained starch is degraded into smaller branched and linear units (maltodextrins) by an alpha-amylase. The liquefaction process is typically carried out at about 105-110° C. for about 5 to 10 minutes followed by about 1-2 hours at about 95° C. The temperature is then lowered to 60° C., a carbohydrate-source generating enzyme, such as a glucoamylase, a beta-amylase, a maltogenic alpha-amylase, or a combination thereof and optionally a debranching enzyme, such as an isoamylase or a pullulanase are added, and the saccharification process proceeds for about 24 to 72 hours.

It will be apparent from the above discussion that the conventional starch conversion process is very energy consuming due to the different requirements in terms of temperature during the various steps. It is thus desirable to be able to select the enzymes used in the process so that the overall process can be performed without having to fully gelatinize the starch. Such processes are the subject for U.S. Pat. Nos. 4,591,560, 4,727,026 and 4,009,074 and EP 171218.

WO03068976 relates to a one-step process for converting granular starch into soluble starch hydrolysate at a temperature below initial gelatinization temperature of the starch.

WO 2013/057141 and WO 2013/057143 describe alpha-amylase variants and uses thereof in, e.g., starch processing, production of fermentation products, processes for producing fermentation products from ungelatinized starch-containing material, and processes for producing fermentation products from gelatinized starch-containing material. These variants are described as having, e.g., increased stability when incubated at low pH and/or at high temperature, in particular at low calcium concentrations, and in particular in the presence of at least 0.1% starch, e.g., in the presence of 0.9% or 1% starch.

There remains a need for improvement of processes for producing starch soluble hydrolysates.

### SUMMARY OF THE INVENTION

In one aspect, the invention provides a process for increasing starch solubilisation comprising subjecting an aqueous granular starch slurry to an alpha-amylase variant comprising an alteration at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1 at a temperature below or just above the initial gelatinization temperature of said granular starch; in the presence of a carbohydrate-source generating enzyme, such as a glucoamylase, a beta-amylase, a maltogenic alpha-amylase, or a combination thereof; thereby increasing solubilisation of the granular starch. In a particular embodiment, the process is performed at a temperature of about 60-70° C.

In one aspect, the invention provides a one step process for producing a soluble starch hydrolysate, the process comprising subjecting a aqueous granular starch slurry at a temperature below or just above the initial gelatinization temperature of said granular starch to the action of an alpha-amylase variant comprising an alteration at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1. In a particular embodiment, the process is performed at a temperature of about 60-70° C.

In a further aspect the invention provides a process for production of high fructose starch-based syrup (HFSS), the process comprising producing a soluble starch hydrolysate by the one step process of the preceding aspect of the invention, and further comprising a step for conversion of the soluble starch hydrolysate into a high fructose starch-based syrup (HFSS).

In a further aspect the invention provides a process for production of maltose syrup, the process comprising producing a soluble starch hydrolysate by the one step process of a preceding aspect of the invention, and further comprising a step for conversion of the soluble starch hydrolysate into a maltose syrup.

### BRIEF DESCRIPTION OF THE FIGURES

FIG. 1 shows an alignment of alpha-amylases with the amino acid sequences of:

SEQ ID NO: 1 is a Bacillus licheniformis alpha-amylase.

SEQ ID NO: 2 is a Bacillus stearothermophilus alpha-amylase.

SEQ ID NO: 3 is the Bacillus alpha-amylase TS-23 described in J. Appl. Microbiology, 1997, 82: 325-334 (SWALL:q59222).

SEQ ID NO: 4 is Bacillus flavothermus alpha-amylase AMY1048 described in WO 2005/001064.

SEQ ID NO: 5 is Bacillus alpha-amylase TS-22 described as SEQ ID NO: 21 in WO 04/113511.

SEQ ID NO: 6 is a Bacillus amyloliquefaciens alpha-amylase.

SEQ ID NO: 7 is Bacillus alkaline sp. SP690 amylase described as SEQ ID NO 1 in WO 95/26397.

SEQ ID NO: 8 is Bacillus halmapalus alpha-amylase described as SEQ ID NO 2 in WO 95/26397.

SEQ ID NO: 9 is Bacillus alkaline sp. AA560 amylase described as SEQ ID NO 4 in WO 00/60060.

SEQ ID NO: 10 is Bacillus alkaline sp. A 7-7 amylase described as SEQ ID NO 2 in WO200210356.

SEQ ID NO: 11 is Bacillus alkaline sp. SP707 amylase described in Tsukamoto et al., 1988, Biochem. Biophys. Res. Commun. 151: 25-33).

SEQ ID NO: 12 is Bacillus alkaline sp. K-38 amylase described as SEQ ID NO 2 in EP 1022334.

SEQ ID NO: 13 is a Bacillus licheniformis alpha-amylase described in Lee et al, 2006, J. Biochem, 139: 997-1005.

SEQ ID NO: 14 is a variant alpha-amylase LE399 previously disclosed in, e.g., WO 2002/010355.

### DETAILED DESCRIPTION OF THE INVENTION

### Definitions

The term “granular starch” is understood as raw uncooked starch, i.e. starch that has not been subjected to a gelatinization. Starch is formed in plants as tiny granules insoluble in water. These granules are preserved in starches at temperatures below the initial gelatinization temperature. When put in cold water, the grains may absorb a small amount of the liquid. Up to 50° C. to 70° C. the swelling is reversible, the degree of reversibility being dependent upon the particular starch. With higher temperatures an irreversible swelling called gelatinization begins.

The term “initial gelatinization temperature” is understood as the lowest temperature at which gelatinization of the starch commences. Starch begins to gelatinize between 60° C. and 70° C., the exact temperature dependent on the specific starch. The initial gelatinization temperature depends on the source of the starch to be processed. The initial gelatinization temperature for wheat starch is approximately 52° C., for potato starch approximately 56° C., and for corn starch approximately 62° C. However, the quality of the starch initial may vary according to the particular variety of the plant species as well as with the growth conditions and therefore initial gelatinization temperature should be determined for each individual starch lot.

The term “soluble starch hydrolysate” is understood as the soluble products of the processes of the invention and may comprise mono- di-, and oligosaccharides, such as glucose, maltose, maltodextrins, cyclodextrins and any mixture of these. Preferably at least 90%, 91%, 92%, 93%, 94%, 95%, 96%, 97% or 98% of the dry solids of the granular starch is converted into a soluble starch hydrolysate.

The term “Speciality Syrups”, is an in the art recognised term and is characterised according to DE and carbohydrate spectrum (See the article “New Speciality Glucose Syrups”, p. 50+, in the textbook “Molecular Structure and Function of Food Carbohydrate”, Edited by G. G. Birch and L. F. Green, Applied Science Publishers LTD., London). Typically Speciality Syrups have a DE in the range from 35 to 45.

Alpha-amylase: Alpha-amylases (E.C. 3.2.1.1) are a group of enzymes which catalyze the hydrolysis of starch and other linear and branched 1,4 glucosidic oligo- and polysaccharides. The skilled person will know how to determine alpha-amylase activity. It may be determined according to the procedure described in the Examples, e.g., by the PNP-G7 assay. In one aspect, the variants of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 1. In another aspect, a variant of the present application has at least 20%, e.g., at least 40%, at least 50%, at least 60%, at least 70%, at least 80%, at least 90%, at least 95%, or at least 100% of the alpha-amylase activity of its parent.

Glucoamylase activity: The term “glucoamylase activity” means 1,4-alpha-D-glucan glucohydrolase activity, (EC 3.2.1.3) that catalyzes the release of D-glucose from the non-reducing ends of starch or related oligo- and polysaccharide molecules. For purposes of the present invention, glucoamylase activity is determined according to the procedure described in the “Materials and Methods”-section below. The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

Beta-amylase activity: The term “beta-amylase activity” means 4-alpha-D-glucan maltohydrolase activity (EC 3.2.1.2), which catalyzes the hydrolysis of (1->4)-alpha-D-glucosidic linkages in polysaccharides so as to remove successive maltose units from the non-reducing ends of the chains until the molecule is degraded or, in the case of amylopectin, until a branch point is reached. The maltose released has the beta anomeric configuration, hence the name beta-amylase. Beta-amylase is the name traditionally given to exo-acting maltogenic amylases, which catalyze the hydrolysis of 1,4-alpha-glucosidic linkages in amylose, amylopectin and related glucose polymers. For purposes of the present invention, beta-amylase activity is determined according to the procedure described in the “Materials and Methods” section below.

Fragment: The term “fragment” means a polypeptide having one or more (e.g., several) amino acids absent from the amino and/or carboxyl terminus of a mature polypeptide; wherein the fragment has alpha-amylase activity. In one aspect, a fragment contains at least 300 amino acid residues, at least 350 amino acid residues, at least 400 amino acid residues, at least 450 amino acid residues, at least 470 amino acid residues, or at least 480 amino acid residues.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two or more different mature polypeptides (i.e., with a different C-terminal and/or N-terminal amino acid) expressed by the same polynucleotide. The mature form of some alpha-amylases, e.g., some bacterial alpha-amylases, comprises a catalytic domain containing the active site for substrate hydrolysis and one or more carbohydrate-binding modules (CBM) for binding to the carbohydrate substrate (starch) and optionally a polypeptide linking the CBM(s) with the catalytic domain, a region of the latter type usually being denoted a “linker”.

Parent or parent alpha-amylase: The term “parent” or “parent alpha-amylase” means an alpha-amylase to which an alteration is made to produce the enzyme variants of the present invention. The parent may be a naturally occurring (wild-type) polypeptide or a variant or fragment thereof.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-5 amino acids, adjacent to the amino acid occupying a position. In one aspect, the variants of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 1. In another aspect, a variant of the present application has at least 20%, e.g., at least 40%, at least 50%, at least 60%, at least 70%, at least 80%, at least 90%, at least 95%, or at least 100% of the alpha-amylase activity of its parent. The alpha-amylase activity may be determined by the PNP-G7 assay described in the Examples.

Wild-type alpha-amylase: The term “wild-type” alpha-amylase means an alpha-amylase expressed by a naturally occurring microorganism, such as a bacterium, yeast, or filamentous fungus found in nature.

Conventions for Designation of Variants: For purposes of the present invention, the mature polypeptide disclosed in SEQ ID NO: 1 is used to determine the corresponding amino acid residue in another alpha-amylase. The amino acid sequence of another alpha-amylase is aligned with the mature polypeptide disclosed in SEQ ID NO: 1, and based on the alignment, the amino acid position number corresponding to any amino acid residue in the mature polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively. In the Examples of the present application, multiple mutations are separated by a space, e.g., G205R S411F representing G205R+S411F.

Deletions.

For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple Alterations.

Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different Alterations.

Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### Processes for Hydrolysis of Starch

In traditional glucose processing, a starch slurry is subjected to primary and secondary liquefaction comprising a pH adjustment and a steam jetting treatment at pH 5.5, 105° C. for 0.1 hr, followed by treatment at 95° C. for 1-2 hours. This is traditionally followed by a step of cooling to 60° C. and pH adjustment to pH 4.0-4.5 for 30-60 hours for a separate saccharification step. Thus, the traditional process requires separate liquefaction and saccharification steps, as well as energy and chemical costs.

In contrast, the starch hydrolysis processes of the present invention provide for a single, lower temperature process, which can reduce energy and chemical costs due to, e.g., lower steam use at lower temperatures, and lower chemical use for pH adjustment, as well as other benefits. In addition, the processes of the present invention can eliminate the need for a separate primary and secondary liquefaction step, as well as a separate carbon column filtration required in traditional processing.

The processes of the present invention can also provide a high degree of starch solubilisation, result in high purity dextrose syrup, and can decrease hydrolysis time for an overall decrease in total processing time. Additional benefits can include lower Maillard products and protein solubilisation in syrup.

The present inventors have surprisingly found that certain bacterial low pH amylases, also referred to herein as alpha-amylase variants, developed for high temperature liquefaction processes and that are active between pH 4.5 to 5.5, show a surprisingly high activity toward raw starch at a temperature below or just above the initial gelatinization temperature of starch, e.g., 60-70° C., such as 60-66° C., which is particularly relevant for corn starch. The activity toward raw starch is shown by increasing starch solubilisation, e.g., in the presence of a carbohydrate-source generating enzyme, such as a glucoamylase, a beta-amylase, a maltogenic alpha-amylase, at low temperature.

As disclosed in, e.g., WO 2013/057141 and WO 2013/057143, incorporated by reference herein, alpha-amylase variants comprising an alteration at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1 substitution show increased stability when incubated at low pH and/or at high temperature, in particular at low calcium concentrations, and in particular in the presence of at least 0.1% starch, e.g., in the presence of 0.9% or 1% starch.

These bacterial amylases enable a single step or combined liquefaction-saccharification process in a cold cook process at e.g., pH 4.5. The cold cook process can replace conventional process of high temperature jetting at 105° C. followed by secondary liquefaction at 95° C. and separate saccharification at 60° C. There are cost savings associated with operating costs due to lower steam usage at lower temperature, lower pH chemical use for pH adjustment from liquefaction to saccharification, and potentially lower use of carbon column and ion exchange columns due to lower Maillard products generated at high temperature, and chemicals added during liquefaction and saccharification steps.

In one aspect, the invention provides a process for increasing starch solubilisation comprising subjecting an aqueous granular starch slurry to an alpha-amylase variant as described herein at a temperature below or just above the initial gelatinization temperature of said granular starch; in the presence of a carbohydrate-source generating enzyme, such as a glucoamylase, a beta-amylase, a maltogenic alpha-amylase or a combination thereof; thereby increasing solubilisation of the granular starch.

In a particular aspect, the alpha-amylase variant is present in an amount greater than about 0.05 mg enzyme protein/g dry solids, such as about 0.06 mg EP/g dry solids, about 0.07 mg EP/g dry solids, about 0.08 mg EP/g dry solids, about 0.09 mg EP/g dry solids, about 0.1 mg EP/g dry solids, about 0.11 mg EP/g dry solids, about 0.12 mg EP/g dry solids, about 0.13 mg EP/g dry solids, about 0.14 mg EP/g dry solids, about 0.15 mg EP/g dry solids, about 0.16 mg EP/g dry solids, about 0.17 mg EP/g dry solids, about 0.18 mg EP/g dry solids, about 0.19 mg EP/g dry solids, about 0.20 mg EP/g dry solids, about 0.21 mg EP/g dry solids, about 0.22 mg EP/g dry solids, about 0.23 mg EP/g dry solids, about 0.24 mg EP/g dry solids, about 0.25 mg EP/g dry solids, about 0.26 mg EP/g dry solids, about 0.27 mg EP/g dry solids, about 0.28 mg EP/g dry solids, about 0.29 mg EP/g dry solids, or about 0.30 mg EP/g dry solids.

In an aspect the invention provides a one step process for producing a soluble starch hydrolysate, the process comprising subjecting an aqueous granular starch slurry at a temperature below or just above the initial gelatinization temperature of said granular starch to the action of an alpha-amylase variant as described herein.

In a further aspect the invention provides a process for production of high fructose starch-based syrup (HFSS), the process comprising producing a soluble starch hydrolysate by the process of the preceding aspect of the invention, and further comprising a step for conversion of the soluble starch hydrolysate into high fructose starch-based syrup (HESS). In some embodiments, dextrose syrup can be produced while starch is being hydrolysed, i.e., in a single step or nearly simultaneous process along with addition of the alpha-amylase variant.

In a further aspect the invention provides a process for production of maltose syrup, the process comprising producing a soluble starch hydrolysate by the process of a preceding aspect of the invention, and further comprising a step for conversion of the soluble starch hydrolysate into maltose syrup. In some embodiments, maltose syrup can be produced while starch is being hydrolysed, i.e., in a single step or nearly simultaneous process along with addition of the alpha-amylase variant.

For example, maltose syrup can be prepared from a 5-20 DE partially hydrolysed starch substrate by saccharification using a maltose-producing enzyme at temperature 50-60° C. and pH 5. Maltogenic enzymes such as beta-amylase extracted from germinated barley, or microbial beta-amylase or fungal alpha amylase derived from Aspergillus oryzae are used to produce a hydrolyzate containing about 40-55 wt % maltose. Higher levels of maltose i.e. 55-85 wt % are produced by saccharification using a combination of a beta-amylase, a maltogenic anzyme and a debranching enzyme such as a pullulanase. In some embodiments, high conversion syrups of 60-70 DE containing intermediate levels of maltose and dextrose are also produced.

The starch slurry to be subjected to the processes of the invention may have 20-55% dry solids granular starch, preferably 25-40% dry solids granular starch, more preferably 30-35% dry solids granular starch.

After being subjected to the processes of the invention at least 85%, 86%, 87%, 88%, 89%, 90%, 91%, 92%, 93%, 94%, 95%, 96%, 97%, 98%, or preferably 99% of the dry solids of the granular starch is converted into a soluble starch hydrolysate.

According to one embodiment of the invention, the processes are conducted at a temperature below the initial gelatinization temperature. In such embodiment, the temperature at which the processes are conducted is at least 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or preferably at least 60° C.

In one embodiment, the processes are conducted at a temperature below or just above the initial gelatinization temperature of starch. In a particular embodiment, the process is performed at a temperature of about 60-70° C., such as about 60-66° C., and in particular at about 60° C., about 61° C., about 62° C., about 63° C., about 64° C., about 65° C. and/or about 66° C. These temperature ranges are particularly relevant where the starch is corn starch. As mentioned, one of skill in the art will recognize that various starches will have an initial gelatinzation temperature that may vary according to the plant species, the particular variety of plant species, and/or to the growth conditions, and the temperature conditions can be adjusted accordingly.

The pH at which the processes of the invention is conducted may in be in the range of 3.0 to 7.0, preferably from 3.5 to 6.0, or more preferably from 4.0-5.0, or preferably from 4.5-5.5, such as about 4.5.

The exact composition of the soluble starch hydrolysate produced according to the invention depends on the combination of enzymes applied as well as the type of granular starch processed. Preferably the soluble hydrolysate is maltose with a purity of at least 85%, 90%, 95.0%, 95.5%, 96.0%, 96.5%, 97.0%, 97.5%, 98.0%, 98.5&, 99.0% or 99.5%. Even more preferably the soluble starch hydrolysate is glucose, and most preferably the starch hydrolysate has a DX (glucose percent of total solubilised dry solids) of at least 85%, 90%, 91.0%, 91.5%, 92.0%, 92.5%, 93.0%, 93.5%, 94.0%, 94.5%, 95.0%, 95.5%, 96.0%, 96.5%, 97.0%, 97.5%, 98.0%, 98.5, 99.0% or 99.5%. Equally contemplated, however, is the process wherein the product of the process of the invention, the soluble starch hydrolysate, is a speciality syrup, such as a speciality syrup containing a mixture of glucose, maltose (DP2), DP3 and DPn for use in the manufacture of ice creams, cakes, candies, canned fruit.

The granular starch to be processed in the processes of the invention may in particular be obtained from tubers, roots, stems, legumes, cereals or whole grain. More specifically the granular starch may be obtained from corns, cobs, wheat, barley, rye, milo, sago, cassava, tapioca, sorghum, rice, peas, bean, banana or potatoes. Specially contemplated are both waxy and non-waxy types of corn and barley. The granular starch to be processed may be a highly refined starch quality, preferably more than 90%, 95%, 97% or 99.5% pure or it may be a more crude starch containing material comprising milled whole grain including non-starch fractions such as germ residues and fibres. The raw material, such as whole grain, is milled in order to open up the structure and allowing for further processing. Two milling processes are preferred according to the invention: wet and dry milling. In dry milling the whole kernel is milled and used. Wet milling gives a good separation of germ and meal (starch granules and protein) and is with a few exceptions applied at locations where the starch hydrolysate is used in production of syrups. Both dry and wet milling are well known in the art of starch processing and are equally contemplated for the processes of the invention. The processes of the invention may be conducted in an ultrafiltration system where the retentate is held under recirculation in presence of enzymes, raw starch and water and where the permeate is the soluble starch hydrolysate. Equally contemplated is the process conducted in a continuous membrane reactor with ultrafiltration membranes and where the retentate is held under recirculation in presence of enzymes, raw starch and water and where the permeate is the soluble starch hydrolysate. Also contemplated is the process conducted in a continuous membrane reactor with microfiltration membranes and where the retentate is held under recirculation in presence of enzymes, raw starch and water and where the permeate is the soluble starch hydrolysate.

In some embodiments, the soluble starch hydrolysate is subjected to conversion into high fructose starch-based syrup (HFSS), such as high fructose corn syrup (HFCS). This conversion is preferably achieved using a glucose isomerase, and more preferably by an immobilized glucose isomerase supported on a solid support. Contemplated isomerases comprises the commercial products Sweetzyme™ IT from Novozymes A/S, G-zyme™ IMGI and G-zyme™ G993, Ketomax™ and G-zyme™ G993 from Rhodia, G-zyme™ G993 liquid and GenSweet™ IGI from Genencor Int.

### Alpha-Amylase Variants

An alpha-amylase variant useful according to the invention is described, e.g., in WO 2013/057141 and WO 2013/057143, incorporated by reference herein.

In particular, alpha-amylase variants comprising an alteration at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1, wherein the variant has at least 60% and less than 100% sequence identity to (i) the mature polypeptide of any of SEQ ID NOs: 1, 2, 3, 4, 5, 6, 7, 8, 9, 10, 11, 12, 13 or 14, or (ii) amino acids 1 to 483 of SEQ ID NO: 1, amino acids 1 to 483 of SEQ ID NO: 2, amino acids 1 to 485 of SEQ ID NO: 3, amino acids 1 to 482 of SEQ ID NO: 4, amino acids 1 to 484 of SEQ ID NO: 5, amino acids 1 to 483 of SEQ ID NO: 6, amino acids 1 to 485 of SEQ ID NO: 7, amino acids 1 to 485 of SEQ ID NO: 8, amino acids 1 to 485 of SEQ ID NO: 9, amino acids 1 to 485 of SEQ ID NO: 10, amino acids 1 to 485 of SEQ ID NO: 11, amino acids 1 to 480 of SEQ ID NO: 12, amino acids 1 to 483 of SEQ ID NO: 13 or amino acids 1 to 481 of SEQ ID NO: 14, and wherein the variant has alpha-amylase activity.

Preferably, the variants are isolated.

Alpha-amylase variants contemplated herein preferably comprise a substitution at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1, wherein the variant has at least 60% and less than 100% sequence identity to (i) the mature polypeptide of any of SEQ ID NOs: 1, 2, 3, 4, 5, 6, 7, 8, 9, 10, 11, 12, 13 or 14, or (ii) amino acids 1 to 483 of SEQ ID NO: 1, amino acids 1 to 483 of SEQ ID NO: 2, amino acids 1 to 485 of SEQ ID NO: 3, amino acids 1 to 482 of SEQ ID NO: 4, amino acids 1 to 484 of SEQ ID NO: 5, amino acids 1 to 483 of SEQ ID NO: 6, amino acids 1 to 485 of SEQ ID NO: 7, amino acids 1 to 485 of SEQ ID NO: 8, amino acids 1 to 485 of SEQ ID NO: 9, amino acids 1 to 485 of SEQ ID NO: 10, amino acids 1 to 485 of SEQ ID NO: 11, amino acids 1 to 480 of SEQ ID NO: 12, amino acids 1 to 483 of SEQ ID NO: 13 or amino acids 1 to 481 of SEQ ID NO: 14, and wherein the variant has alpha-amylase activity.

In one embodiment, the variant comprises one or more alterations selected from the group consisting of A1AH, A1AF, A1AY, A1AW, A1H, A1F, A1Y, A1W, N2NH, N2N2F, N2NY, N2NW, N2H, N2F, N2Y, N2W, H68F, H68Y, H68W, G71F, G71H, G71Y, G71W, N126F, N126H, N126Y, N126W, H133F, H133Y, H133W, H142F, H142Y, H142W, P144F, P144H, P144Y, P144W, Y156F, Y156H, Y156W, Y158F, Y158H, Y158W, K176L, E185P, I120F, I201Y, H205Y, K213T, S239A, S239Q, F279Y, F279W, H316F, H316Y, H316W, L318F, L318H, L318Y, L318W, Q360S, D416V, R437F, R437H, R437Y, R437W, H450F, H450Y and H450W.

In a preferred embodiment, the variant comprises one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V, R437W and H450W.

In another preferred embodiment, the variant comprises two or more substitutions selected from the group consisting of A1N, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V, R437W and H450W.

In another embodiment, the variant comprises three or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V, R437W and H450W. In another embodiment, the variant comprises four or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V, R437W and H450W.

In one embodiment, the variant comprises a substitution at a position corresponding to position 176, in particular the substitution K176L, in combination with an alteration at one or more positions corresponding to any of positions 1, 2, 68, 71, 126, 133, 142, 144, 156, 158, 185, 201, 205, 213, 239, 279, 316, 318, 360, 416, 437 and 450, in particular one or more alterations selected from the group consisting of A1AH, A1AW, A1H, A1W, N2NH, N2NW, N2H, N2W, H68W, G71W, N126W, H133Y, H142W, P144W, Y156W, Y158W, E185P, I201Y, H205Y, K213T, S239A, S239Q, F279W, H316W, L318W, Q360S, D416V, R437W and H450W.

In a preferred embodiment, the variant comprises a substitution at a position corresponding to position 176, in particular the substitution K176L, in combination with an alteration at one or more positions corresponding to any of positions 1, 2, 68, 71, 126, 133, 142, 144, 156, 158, 185, 201, 205, 213, 239, 279, 316, 318, 360, 416, 437 and 450, in particular one or more alterations selected from the group consisting of A1AH, A1AW, A1H, A1W, N2NH, N2NW, N2H, N2W, H68W, G71W, N126W, H133Y, H142W, P144W, Y156W, Y158W, E185P, I201Y, H205Y, K213T, S239A, S239Q, F279W, H316W, L318W, Q360S, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 1, or (ii) amino acids 1-483 of SEQ ID NO: 1.

In a preferred embodiment, the variant comprises a substitution at a position corresponding to position 176, in particular the substitution K176L, in combination with an alteration at one or more positions corresponding to any of positions 1, 2, 68, 71, 126, 133, 142, 144, 156, 158, 185, 201, 205, 213, 239, 279, 316, 318, 360, 416, 437 and 450, in particular one or more alterations selected from the group consisting of A1AH, A1AW, A1H, A1W, N2NH, N2NW, N2H, N2W, H68W, G71W, N126W, H133Y, H142W, P144W, Y156W, Y158W, E185P, I201Y, H205Y, K213T, S239A, S239Q, F279W, H316W, L318W, Q360S, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 14, or (ii) amino acids 1-481 of SEQ ID NO: 14.

In one embodiment, the variant comprises a substitution at a position corresponding to position 185, in particular the substitution E185P, in combination with a substitution at one or more positions corresponding to any of positions 1, 2, 68, 71, 126, 133, 142, 144, 156, 158, 176, 201, 205, 213, 239, 279, 316, 318, 360, 416, 437 and 450, in particular one or more substitutions selected from the group consisting of A1H, A1W, N2H, N2W, H68W, G71W, N126W, H133Y, H142W, P144W, Y156W, Y158W, K176L, I201Y, H205Y, K213T, S239A, S239Q, F279W, H316W, L318W, Q360S, D416V, R437W and H450W.

In a preferred embodiment, the variant comprises a substitution at a position corresponding to position 185, in particular the substitution E185P, in combination with a substitution at one or more positions corresponding to any of positions 1, 2, 68, 71, 126, 133, 142, 144, 156, 158, 176, 201, 205, 213, 239, 279, 316, 318, 360, 416, 437 and 450, in particular one or more substitutions selected from the group consisting of A1H, A1W, N2H, N2W, H68W, G71W, N126W, H133Y, H142W, P144W, Y156W, Y158W, K176L, I201Y, H205Y, K213T, S239A, S239Q, F279W, H316W, L318W, Q360S, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 1, or (ii) amino acids 1-483 of SEQ ID NO: 1.

In a preferred embodiment, the variant comprises a substitution at a position corresponding to position 185, in particular the substitution E185P, in combination with a substitution at one or more positions corresponding to any of positions 1, 2, 68, 71, 126, 133, 142, 144, 156, 158, 176, 201, 205, 213, 239, 279, 316, 318, 360, 416, 437 and 450, in particular one or more substitutions selected from the group consisting of A1H, A1W, N2H, N2W, H68W, G71W, N126W, H133Y, H142W, P144W, Y156W, Y158W, K176L, I201Y, H205Y, K213T, S239A, S239Q, F279W, H316W, L318W, Q360S, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 14, or (ii) amino acids 1-481 of SEQ ID NO: 14.

In one embodiment, the variant comprises a substitution at a position corresponding to position 360, in particular the substitution Q360S, in combination with a substitution at one or more positions corresponding to any of positions 1, 2, 68, 71, 126, 133, 142, 144, 156, 158, 176, 185, 201, 205, 213, 239, 279, 316, 318, 416, 437 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, D416V, R437W and H450W.

In a preferred embodiment, the variant comprises a substitution at a position corresponding to position 360, in particular the substitution Q360S, in combination with a substitution at one or more positions corresponding to any of positions 1, 2, 68, 71, 126, 133, 142, 144, 156, 158, 176, 185, 201, 205, 213, 239, 279, 316, 318, 416, 437 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 1, or (ii) amino acids 1-483 of SEQ ID NO: 1.

In a preferred embodiment, the variant comprises a substitution at a position corresponding to position 360, in particular the substitution Q360S, in combination with a substitution at one or more positions corresponding to any of positions 1, 2, 68, 71, 126, 133, 142, 144, 156, 158, 176, 185, 201, 205, 213, 239, 279, 316, 318, 416, 437 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, D416V, R437W and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 14, or (ii) amino acids 1-481 of SEQ ID NO: 14.

In one embodiment, the variant comprises a substitution at a position corresponding to position 437, in particular the substitution R437W, in combination with a substitution at one or more positions corresponding to any of positions 1, 2, 68, 71, 126, 133, 142, 144, 156, 158, 176, 185, 201, 205, 213, 239, 279, 316, 318, 360, 416 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V and H450W.

In a preferred embodiment, the variant comprises a substitution at a position corresponding to position 437, in particular the substitution R437W, in combination with a substitution at one or more positions corresponding to any of positions 1, 2, 68, 71, 126, 133, 142, 144, 156, 158, 176, 185, 201, 205, 213, 239, 279, 316, 318, 360, 416 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 1, or (ii) amino acids 1-483 of SEQ ID NO: 1.

In a preferred embodiment, the variant comprises a substitution at a position corresponding to position 437, in particular the substitution R437W, in combination with a substitution at one or more positions corresponding to any of positions 1, 2, 68, 71, 126, 133, 142, 144, 156, 158, 176, 185, 201, 205, 213, 239, 279, 316, 318, 360, 416 and 450, in particular one or more substitutions selected from the group consisting of A1H, A1W, N2H, N2W, H68W, G71W, N126W, H133Y, H142W, P144W, Y156W, Y158W, K176L, E185P, I201Y, H205Y, K213T, S239A, S239Q, F279W, H316W, L318W, Q360S, D416V and H450W, and the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to (i) the mature polypeptide of SEQ ID NO: 14, or (ii) amino acids 1-481 of SEQ ID NO: 14.

In a preferred embodiment, a variant comprises a set of substitutions selected from the group consisting of:

A1H+N2W+K176L+E185P,

A1W+N2H+K176L+E185P,

N2H+H68W+H133Y+K176L+E185P,

N2H+H68W+Y156W+K176L+E185P,

N2H+H68W+Y158W+K176L+E185P,

N2H+H68W+K176L+E185P,

N2H+H68W+K176L+E185P+I201Y+H205Y+D207V+V209D,

N2H+H68W+K176L+E185P+F279W,

N2H+H133Y+K176L+E185P+H316W+R437W,

N2H+H133Y+K176L+E185P+Q360S+R437W,

N2H+H142W+K176L+E185P+H316W+R437W,

N2H+H142W+K176L+E185P+Q360S+R437W,

N2H+P144W+K176L+E185P,

N2H+Y156W+Y158W+K176L+E185P+H316W+R437W,

N2H+Y156W+K176L+E185P+Q360S+R437W,

N2H+Y158W+K176L+E185P+I201Y+H205Y+D207V+V209D+H316W,

N2H+K176L+E185P,

N2H+K176L+E185P+H316W,

N2H+K176L+E185P+H316W+L318W+R437W,

N2H+K176L+E185P+H316W+Q360S+R437W,

N2H+K176L+E185P+H316W+R437W,

N2H+K176L+E185P+R437W,

N2H+K176L+E185P+O360S+R437W,

N2H+K176L+E185P+H316W+Q360S+R437W,

N2H+K176L+I201Y+H205Y+K213T+Q360S+D416V+R437W,

H68W+K176L+E185P,

H68W+K176L+E185P+I201Y+H205Y+K213T+Q360S+D416V+R437W,

H68W+K176L+I201Y+H205Y+K213T+Q360S+D416V+R437W,

G71W+K176L+E185P,

N126W+K176L+I201Y+H205Y+K213T+O360S+D416V+R437W,

H133Y+Y158W+K176L+E185P+I201Y+H205Y+K213T+O360S+D416V+R437W,

H133Y+K176L+I201Y+H205Y+K213T+O360S+D416V+R437W,

H142W+Y158W+K176L+E185P+I201Y+H205Y+K213T+Q360S+D416V+R437W,

H142W+K176L+E185P,

H142W+K176L+E185P+I201Y+H205Y+K213T+Q360S+D416V+R437W,

H142W+K176L+I201Y+H205Y+K213T+Q360S+D416V+R437W,

P144W+K176L+E185P,

Y156W+Y158W+K176L+E185P+I201Y+H205Y+K213T+O360S+D416V+R437W,

Y156W+Y158W+K176L+E185P+H316W+R437W,

Y156W+K176L+E185P+O360S+R437W,

Y156W+K176L+I201Y+H205Y+K213T+O360S+D416V+R437W,

Y158W+K176L+E185P,

Y158W+K176L+E185P+I201Y+H205Y+D207V+V209D+H316W,

Y158W+K176L+E185P+I201Y+H205Y+K213T+H316L+L318W+Q360S+D416V+R437,

Y158W+K176L+E185P+I201Y+H205Y+K213T+H316W+Q360S+D416V+R437W,

Y158W+K176L+E185P+I201Y+H205Y+K213T+Q360S+D416V+R437W,

Y158W+K176L+I201Y+H205Y+K213T+O360S+D416V+R437W,

K176L+E185P,

K176L+E185P+I201Y+H205Y+K213T+O360S+D416V+R437W,

K176L+E185P+I201Y+H205Y+R437W,

K176L+E185P+F279W,

K176L+E185P+H316W,

K176L+E185P+L318W,

K176L+E185P+H450W,

K176L+I201Y+H205Y+K213T+S239Q+Q360S+D416V+R437W,

K176L+I201Y+H205Y+K213T+H316W+O360S+D416V+R437W,

K176L+I201Y+H205Y+K213T+L318W+Q360S+D416V+R437W,

K176L+I201Y+H205Y+K213T+O360S+D416V+R437W,

K176L+I201Y+H205Y+K213T+Q360S+R437W,

K176L+I201Y+H205Y+K213T+D416V+R437W, and

K176L+I201Y+H205Y+Q360S+D416V+R437W.

In another preferred embodiment, the variant comprises a set of substitutions selected from the group consisting of:

A1H+N2W+K176L+E185P,

A1W+N2H+K176L+E185P,

N2H+H68W+H133Y+K176L+E185P,

N2H+H68W+Y156W+K176L+E185P,

N2H+H68W+Y158W+K176L+E185P,

N2H+H68W+K176L+E185P,

N2H+H68W+K176L+E185P+I201Y+H205Y+D207V+V209D,

N2H+H68W+K176L+E185P+F279W,

N2H+H133Y+K176L+E185P+H316W+R437W,

N2H+H133Y+K176L+E185P+Q360S+R437W,

N2H+H142W+K176L+E185P+H316W+R437W,

N2H+H142W+K176L+E185P+Q360S+R437W,

N2H+P144W+K176L+E185P,

N2H+Y156W+Y158W+K176L+E185P+H316W+R437W,

N2H+Y156W+K176L+E185P+Q360S+R437W,

N2H+Y158W+K176L+E185P+I201Y+H205Y+D207V+V209D+H316W,

N2H+K176L+E185P,

N2H+K176L+E185P+H316W,

N2H+K176L+E185P+H316W+L318W+R437W,

N2H+K176L+E185P+H316W+Q360S+R437W,

N2H+K176L+E185P+H316W+R437W,

N2H+K176L+E185P+R437W,

N2H+K176L+E185P+Q360S+R437W,

N2H+K176L+E185P+H316W+Q360S+R437W,

N2H+K176L+I201Y+H205Y+K213T+O360S+D416V+R437W,

H68W+K176L+E185P,

H68W+K176L+E185P+I201Y+H205Y+K213T+O360S+D416V+R437W,

H68W+K176L+I201Y+H205Y+K213T+O360S+D416V+R437W,

G71W+K176L+E185P,

N126W+K176L+I201Y+H205Y+K213T+O360S+D416V+R437W,

H133Y+Y158W+K176L+E185P+I201Y+H205Y+K213T+Q360S+D416V+R437W,

H133Y+K176L+I201Y+H205Y+K213T+Q360S+D416V+R437W,

H142W+Y158W+K176L+E185P+I201Y+H205Y+K213T+Q360S+D416V+R437W,

H142W+K176L+E185P,

H142W+K176L+E185P+I201Y+H205Y+K213T+Q360S+D416V+R437W,

H142W+K176L+I201Y+H205Y+K213T+Q360S+D416V+R437W,

P144W+K176L+E185P,

Y156W+Y158W+K176L+E185P+I201Y+H205Y+K213T+O360S+D416V+R437W,

Y156W+Y158W+K176L+E185P+H316W+R437W,

Y156W+K176L+E185P+Q360S+R437W,

Y156W+K176L+I201Y+H205Y+K213T+Q360S+D416V+R437W,

Y158W+K176L+E185P,

Y158W+K176L+E185P+I201Y+H205Y+D207V+V209D+H316W,

Y158W+K176L+E185P+I201Y+H205Y+K213T+H316L+L318W+O360S+D416V+R437W,

Y158W+K176L+E185P+I201Y+H205Y+K213T+H316W+O360S+D416V+R437W,

Y158W+K176L+E185P+I201Y+H205Y+K213T+O360S+D416V+R437W,

Y158W+K176L+I201Y+H205Y+K213T+O360S+D416V+R437W,

K176L+E185P,

K176L+E185P+I201Y+H205Y+K213T+Q360S+D416V+R437W,

K176L+E185P+I201Y+H205Y+R437W,

K176L+E185P+F279W,

K176L+E185P+H316W,

K176L+E185P+L318W,

K176L+E185P+H450W,

K176L+I201Y+H205Y+K213T+S239O+O360S+D416V+R437W,

K176L+I201Y+H205Y+K213T+H316W+Q360S+D416V+R437W,

K176L+I201Y+H205Y+K213T+L318W+Q360S+D416V+R437W,

K176L+I201Y+H205Y+K213T+Q360S+D416V+R437W,

K176L+I201Y+H205Y+K213T+Q360S+R437W,

K176L+I201Y+H205Y+K213T+D416V+R437W, and

K176L+I201Y+H205Y+O360S+D416V+R437W.

In a preferred embodiment, the variant comprises a set of substitutions selected from the group consisting of:

T49H+K176L+E185P,

T49G+K176L+E185P,

T49L+S50T+K176L+E185P,

T116G+K176L+E185P,

K176L+E185P,

K176L+E185P+I201Y+H205Y+K213T+Q360S+D416V+R437W,

K176L+E185P+L241D,

K176L+E185P+R375V, and

K176L+E185P+R375G.

In another preferred embodiment, the variant comprises a set of substitutions selected from the group consisting of:

G48A+T49H+G107A+H156Y+K176L+A181T+E185P+N190F+I201F+A209V+Q264S;

G48A+T49G+G107A+H156Y+K176L+A181T+E185P+N190F+I201F+A209V+Q264S;

G48A+T49L+S50T+G107A+H156Y+K176L+A181T+E185P+N190F+I201F+A209V+Q264S;

G48A+T49I+G107A+T116G+H156Y+K176L+A181T+E185P+N190F+I201F+A209V+Q264S;

G48A+T49I+G107A+H156Y+K176L+A181T+E185P+N190F+I201F+A209V+O264S;

G48A+T49I+G107A+H156Y+K176L+A181T+E185P+N190F+I201Y+H205Y+A209V+K213T+Q264S+Q360S+D416V+R437W;

G48A+T49I+G107A+H156Y+K176L+A181T+E185P+N190F+I201F+L241D+A209V+Q264S;

G48A+T49I+G107A+H156Y+K176L+A181T+E185P+N190F+I201F+A209V+Q264S+R375V;

G48A+T49I+G107A+H156Y+K176L+A181T+E185P+N190F+I201F+A209V+O264S+R375G; and

G48A+G107A+H156Y+K176L+A181T+E185P+N190F+I201F+A209V+Q264S.

In one embodiment, the variant comprises a substitution at position 176 and/or 185. Preferably the substitution is 176+185, and more preferably K176L+E185P.

In one embodiment, the variant comprises a substitution at one or more of positions 176, 185, 360 and/or 437. Preferably the substitution is 176+185+360+437, more preferably K176L+E185P+Q360S+R437W.

In one embodiment, the variant further comprises a deletion at both of the two positions immediately before the position corresponding to position 180 of SEQ ID NO: 1. I.e., a deletion of the two amino acids corresponding to positions 181 and 182 of SEQ ID NO: 2.

In another embodiment, the variant further comprises a deletion of two amino acids after the position corresponding to position 177 of SEQ ID NO: 1 and before the position corresponding to position 180 of SEQ ID NO: 1. I.e., a deletion of two amino acids in the R179-G180-I181-G182 peptide of SEQ ID NO: 2, or homologous amino acids in any of SEQ ID NO: 3 to 11. The variants may further comprise one or more (e.g., several) additional alterations, e.g., one or more (e.g., several) additional substitutions.

The additional amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants may consist of 300 to 700, e.g., 350 to 650, 400 to 600, 450 to 500 or 470 to 490, amino acids.

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used.

### Glucoamylase

In some embodiments, the processes of the invention comprise addition of a glucoamylase. The glucoamylase (E.C.3.2.1.3) may be derived from a microorganism or a plant.

In some embodiments, the glucoamylase has a starch binding domain.

Preferred are Trametes glucoamylases, such as glucoamylase from Trametes cingulata (WO 2006/069289), or variants or fragments thereof.

Also preferred are Gloeophyllum glucoamylases, such as glucoamylase from the Gloeophillum sp. selected from the group consisting of G. abietinum, G. sepiarium, and G. trabeum, and in particular Gloeophyllum trabeum (WO 2011/068803), or variants or fragments thereof. Also preferred are variants of Gloeophyllum species, as disclosed in, e.g., application number EP13165995 and in particular the variant having the double substitution S95P, A121P.

Exemplary glucoamylase of fungal or bacterial origin selected from the group consisting of Aspergillus glucoamylases, in particular A. niger G1 or G2 glucoamylase (Boel et al., 1984, EMBO J. 3 (5): 1097-1102), or variants thereof, such as disclosed in WO 92/00381 and WO 00/04136; the A. awamori glucoamylase (WO 84/02921), A. oryzae (Agric. Biol. Chem., 1991, 55(4): 941-949), or variants or fragments thereof.

Other contemplated Aspergillus glucoamylase variants include variants to enhance the thermal stability: G137A and G139A (Chen et al., 1996, Prot. Engng. 9: 499-505); D257E and D293E/Q (Chen et al., 1995, Prot. Engng. 8: 575-582); N182 (Chen et al., 1994, Biochem. J. 301: 275-281); disulphide bonds, A246C (Fierobe et al., 1996, Biochemistry, 35: 8698-8704; and introduction of Pro residues in position A435 and S436 (Li et al., 1997, Protein Engng. 10: 1199-1204. Furthermore Clark Ford presented a paper on Oct. 17, 1997, ENZYME ENGINEERING 14, Beijing/China Oct. 12-17, 1997, Abstract book p. 0-61. The abstract suggests mutations in positions G137A, N20C/A27C, and S30P in an Aspergillus awamori glucoamylase to improve the thermal stability.

Other contemplated glucoamylases include Talaromyces glucoamylases, in particular derived from Talaromyces emersonii (WO 99/28448), Talaromyces leycettanus (U.S. Pat. No. RE 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135138), and C. thermohydrosulfuricum (WO 86/01831). Preferred glucoamylases include the glucoamylases derived from Aspergillus oryzae. Also contemplated are the commercial products AMG 200L; AMG 300 L; SAN™ SUPER and AMG™ E (from Novozymes); OPTIDEX™ 300 (from Genencor Int.); AMIGASE™ and AMIGASE™ PLUS (from DSM); G-ZYME™ G900 (from Enzyme Bio-Systems); G-ZYME™ G990 ZR (A. niger glucoamylase and low protease content).

Glucoamylases may be added in an amount of 0.02-2.0 AGU/g DS, preferably 0.1-1.0 AGU/g DS, such as 0.2 AGU/g DS, or in other effective amounts well known to the person skilled in the art.

### Beta-Amylase

In some embodiments, the processes of the invention comprise addition of a beta-amylase (E.C 3.2.1.2). Beta-amylase is the name traditionally given to exo-acting maltogenic amylases, which catalyze the hydrolysis of 1,4-alpha-glucosidic linkages in amylose, amylopectin and related glucose polymers.

Beta-amylases have been isolated from various plants and microorganisms (W. M. Fogarty and C. T. Kelly, 1979, Progress in Industrial Microbiology, 15: 112-115). These beta-amylases are characterized by having optimum temperatures in the range from 40° C. to 65° C. and optimum pH in the range from 4.5 to 7.0. Contemplated beta-amylases include the beta-amylase from barley Spezyme® BBA 1500, Spezyme® DBA and Optimalt™ ME, Optimalt™ BBA from Genencor Int. as well as Novozym™ WBA from Novozymes NS.

The beta-amylase may also be a microbial beta-amylase, including a beta-amylase derived from Bacillus flexus as described in U.S. Pat. No. 8,486,682.

Beta-amylases can be added in effective amounts well known to the person skilled in the art. The recommended range is 0.05-1 BAMU/gDS.

### Maltogenic Alpha-Amylase

Another particular enzyme to be used as a first enzyme in the processes of the invention is a maltogenic alpha-amylase (E.C. 3.2.1.133). Maltogenic alpha-amylases (glucan 1,4-alpha-maltohydrolase) are able to hydrolyse amylose and amylopectin to maltose in the alpha-configuration. Furthermore, a maltogenic alpha-amylase is able to hydrolyse maltotriose as well as cyclodextrins. Specifically contemplated maltogenic alpha-amylases may be derived from Bacillus sp., preferably from Bacillus stearothermophilus, most preferably from Bacillus stearothermophilus C599 such as the one described in EP120.693. This particular maltogenic alpha-amylase has the amino acid sequence shown as amino acids 1-686 of SEQ ID NO:1 in U.S. Pat. No. 6,162,628. A preferred maltogenic alpha-amylase has an amino acid sequence having at least 70% identity to amino acids 1-686 of SEQ ID NO:1 in U.S. Pat. No. 6,162,628, preferably at least 80%, at least 85%, at least 90%, at least 92%, at least 95%, at least 96%, at least 97%, at least 98%, or particularly at least 99%. Most preferred variants of the maltogenic alpha-amylase comprise the variants disclosed in WO99/43794.

The maltogenic alpha-amylase having the amino acid sequence shown as amino acids 1-686 of Sequence Number 1 in U.S. Pat. No. 6,162,628 a hydrolysis activity of 714. Preferably the maltogenic alpha-amylase to be applied as a first enzyme of the process has a hydrolysis activity of at least 3.5, preferably at least 4, 5, 6, 7, 8, 9, 10, 11, 12, 13, 14, 15, 16, 17, 18, 19, 20, 21, 22, 23, 100, 200, 300, 400, 500, 600, or most preferably at least 700 micro mol per min/mg.

Maltogenic alpha-amylases may be added in amounts of 0.01-40.0 MANU/g DS, preferably from 0.02-10 MANU/g DS, preferably 0.05-5.0 MANU/g DS, or in other effective amounts well known to the person skilled in the art. Contemplated maltogenic alpha-amylases include Novamyl® (Novozymes NS).

### Additional Enzymes

In some embodiments, invention optionally comprises additional enzymes.

### Cyclodextrin Glucanotransferases (CGTases)

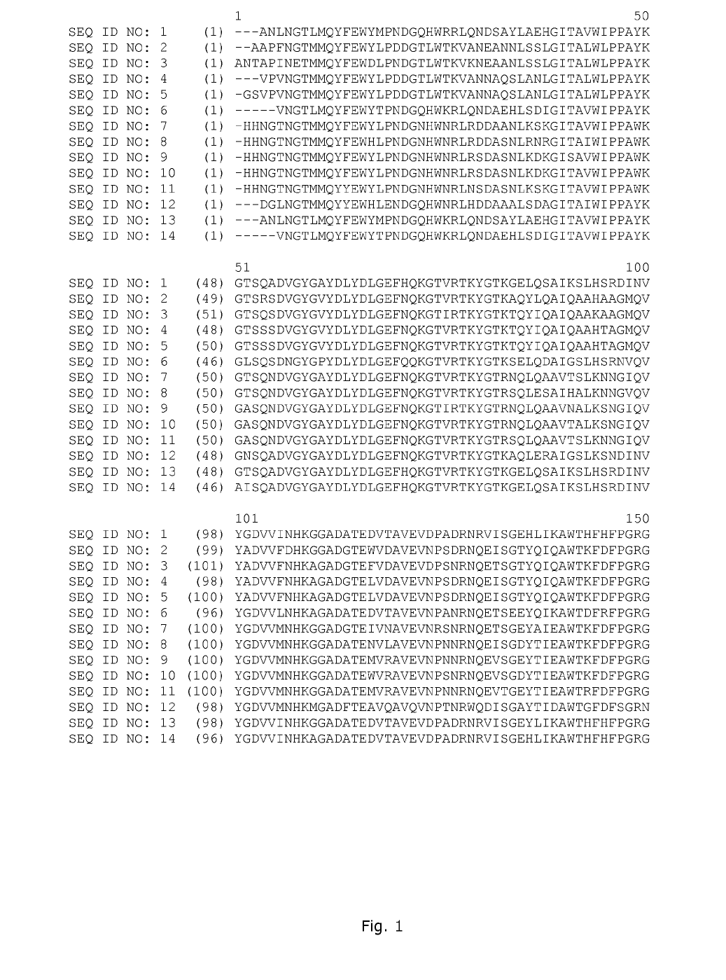

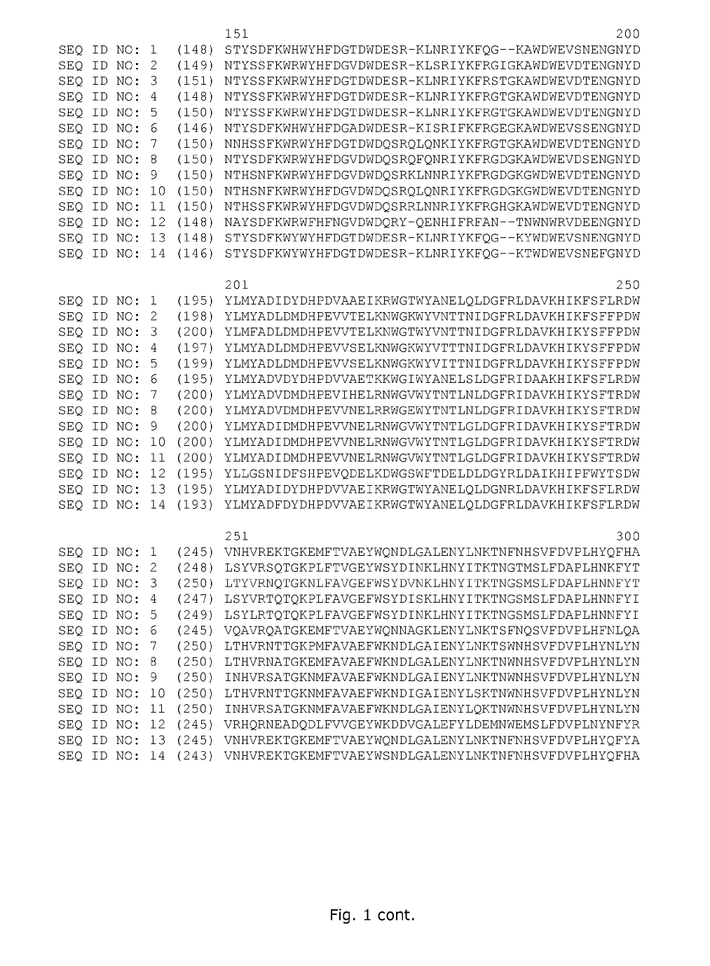

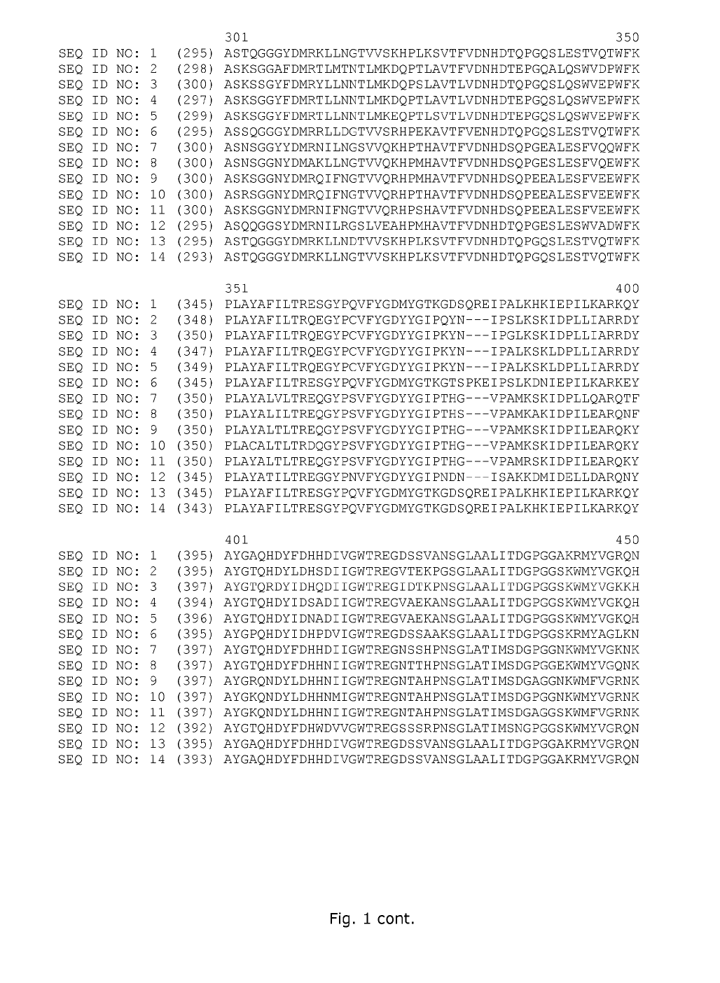

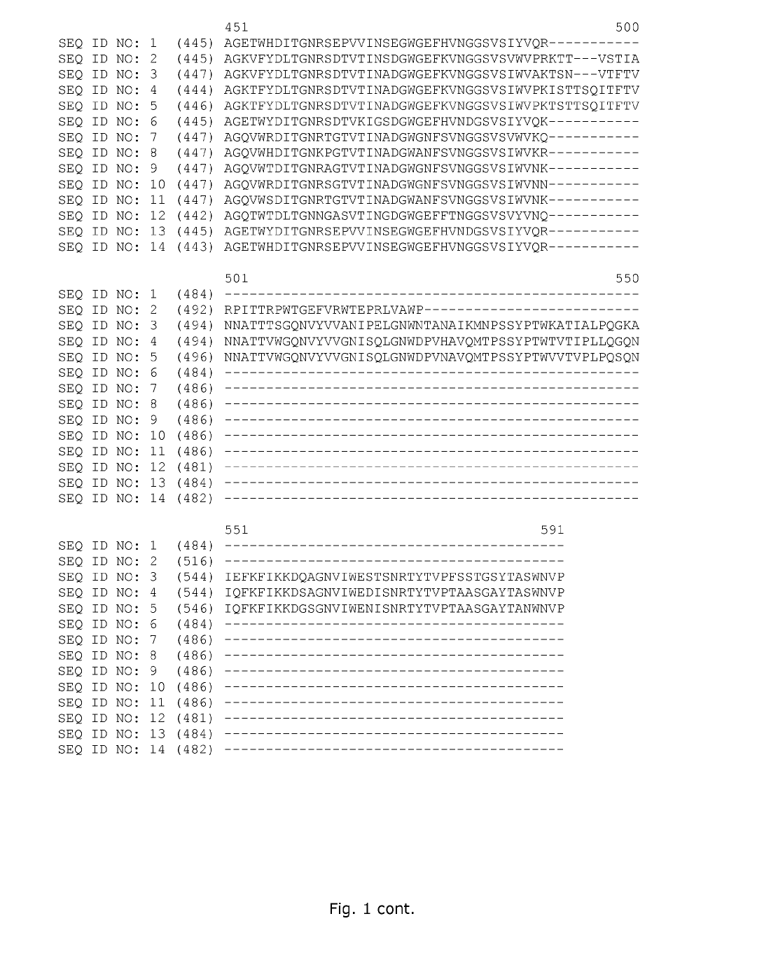

A particular enzyme to be used as a first enzyme in the processes of the invention may be a cyclomaltodextrin glucanotransferase (E.C. 2.4.1.19). Cyclomaltodextrin glucanotransferase, also designated cyclodextrin glucanotransferase or cyclodextrin glycosyltransferase, in the following termed CGTase, catalyses the conversion of starch and similar substrates into cyclomaltodextrins via an intramolecular transglycosylation reaction, thereby forming cyclomaltodextrins of various sizes. Most CGTases have both transglycosylation activity and starch-degrading activity. Contemplated CGTases are preferably of microbial origin, and most preferably of bacterial origin. Specifically contemplated CGTases include the CGTases having 50%, 55%, 60%, 65%, 70%, 75%, 80%, 85% or even 90% homology to the sequence shown as amino acids 1 to 679 of SEQ ID NO:2 in WO02/06508, the CGTases having 50%, 55%, 60%, 65%, 70%, 75%, 80%, 85% or even 90% homology to the amino acid sequence of the polypeptide disclosed in Joergensen et al, 1997 in FIG. 1 in Biotechnol. Lett. 19:1027-1031, and the CGTases described in U.S. Pat. No. 5,278,059 and U.S. Pat. No. 5,545,587. Preferably the CGTase to be applied as a first enzyme of the process has a hydrolysis activity of at least 3.5, preferably at least 4, 4.5, 5, 6, 7, 8, 9, 10, 11, 12, 13, 14, 15, 16, 17, 18, 19, 20, 21, 22, or most preferably at least 23 micro mol per min/mg. CGTases may be added in amounts of 0.01-100.0 NU/g DS, preferably from 0.2-50.0 NU/g DS, preferably 10.0-20.0 NU/g DS, or in other effective amounts well known to the person skilled in the art. The commercial product Toruzyme® (Novozymes NS) is contemplated.

### Fungal Aloha-Amylase

A particular enzyme to be used as additional enzyme in the processes of the invention is a fungal alpha-amylase (EC 3.2.1.1), such as a fungamyl-like alpha-amylase. In the present disclosure, the term “fungamyl-like alpha-amylase” indicates an alpha-amylase which exhibits a high homology, i.e. more than 50%, 55%, 60%, 65%, 70%, 75%, 80%, 85% or even 90% homology to the amino acid sequence of SEQ ID NO: 7. Fungal alpha-amylases may be added in an amount of 0.001-1.0 AFAU/g DS, preferably from 0.002-0.5 AFAU/g DS, preferably 0.02-0.1 AFAU/g DS, or in other effective amounts well known to the person skilled in the art.

### Bacillus Alpha-Amylase

A Bacillus alpha-amylase (often referred to as “Termamyl-like alpha-amylases”). Well-known Termamyl-like alpha-amylases include alpha-amylase derived from a strain of B. licheniformis (commercially available as Termamyl), B. amyloliquefaciens, and B. stearothermophilus alpha-amylase. Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., 1988, Biochemical and Biophysical Research Communications, 151: 25-31. In the context of the present invention a Termamyl-like alpha-amylase is an alpha-amylase as defined in WO 99/19467 on page 3, line 18 to page 6, line 27. Contemplated variants and hybrids are described in WO 96/23874, WO 97/41213, and WO 99/19467. Specifically contemplated is a recombinant B. stearothermophilus alpha-amylase variant with the mutations: I181*+G182*+N193F. Bacillus alpha-amylases may be added in effective amounts well known to the person skilled in the art.

### Debranching Enzymes

Another particular enzyme of the process may be a debranching enzyme, such as an isoamylase (E.C. 3.2.1.68) or a pullulanases (E.C. 3.2.1.41). Isoamylase hydrolyses alpha-1,6-D-glucosidic branch linkages in amylopectin and beta-limit dextrins and can be distinguished from pullulanases by the inability of isoamylase to attack pullulan, and by the limited action on alpha-limit dextrins. Debranching enzyme may be added in effective amounts well known to the person skilled in the art.

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Specifically contemplated pullulanases useful according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans pullulanase disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

The pullulanase may according to the invention be added in an effective amount which include the preferred range of from between 1-100 micro g per g DS, especially from 10-60 micro g per g DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300, and AMANO 8 (Amano, Japan).

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### Materials and Methods

### Assays for Measurement of Amylolytic Activity (Alpha-Amylase Activity)

PNP-G7 Assay:

The alpha-amylase activity is determined by a method employing the PNP-G7 substrate. PNP-G7 is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-glucosidase included in the kit digest the hydrolysed substrate further to liberate a free PNP molecule which has a yellow color and thus can be measured by visible spectophometry at λ=405 nm (400-420 nm.). Kits containing PNP-G7 substrate and alpha-glucosidase is manufactured by Roche/Hitachi (cat. No. 11876473).

Reagents:

The G7-PNP substrate from this kit contains 22 mM 4,6-ethylidene-G7-PNP and 52.4 mM HEPES (2-[4-(2-hydroxyethyl)-1-piperazinyl]-ethanesulfonic acid), pH 7.0.

The alpha-glucosidase reagent contains 52.4 mM HEPES, 87 mM NaCl, 12.6 mM MgCl₂, 0.075 mM CaCl₂, >4 kU/L alpha-glucosidase.

The substrate working solution is made by mixing 1 ml of the alpha-glucosidase reagent with 0.2 ml of the G7-PNP substrate. This substrate working solution is made immediately before use.

Dilution buffer: 50 mM EPPS, 0.01% (w/v) Triton X100 (polyethylene glycol p-(1,1,3,3-tetramethylbutyl)-phenyl ether (C₁₄H₂₂O(C₂H₄O), (n=9-10))), 1 mM CaCl₂, pH 7.0.

Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

Phadebas Activity Assay:

The alpha-amylase activity can also be determined by a method using the Phadebas substrate (from for example Magle Life Sciences, Lund, Sweden). A Phadebas tablet includes interlinked starch polymers that are in the form of globular microspheres that are insoluble in water. A blue dye is covantly bound to these microspheres. The interlinked starch polymers in the microsphere are degraded at a speed that is proportional to the alpha-amylase activity. When the alpha-amylse degrades the starch polymers, the released blue dye is water soluble and concentration of dye can be determined by measuring absorbance at 620 nm. The concentration of blue is proportional to the alpha-amylase activity in the sample.

The amylase sample to be analysed is diluted in dilution buffer with the desired pH. One substrate tablet is suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl 1M NaOH and mix. Centrifuge MTP for 5 minutes at 4000×g. Transfer 100 μl to new MTP and measure absorbance at 620 nm.

The amylase sample should be diluted so that the absorbance at 620 nm is between 0 and 2.2.

EnzChek® Assay:

For the determination of residual amylase activity an EnzChek® Ultra Amylase Assay Kit (E33651, Invitrogen, La Jolla, Calif., USA) was used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM acetate, 0.01% (w/v) TRITON® X100, 0.125 mM CaCl₂, pH 5.5 is added, vortexed thoroughly and stored at room temperature, in the dark until ready to use. The stock substrate working solution is prepared by diluting 10-fold in residual activity buffer (100 mM acetate, 0.01% (w/v) TRITON® X100, 0.125 mM CaCl₂, pH 5.5). Immediately after incubation the enzyme is diluted to a concentration of 10-20 ng enzyme protein/ml in 100 mM acetate, 0.01% (W/v) TRITON® X100, 0.125 mM CaCl₂, pH 5.5.

For the assay, 25 microliters of the substrate working solution is mixed for 10 second with 25 microliters of the diluted enzyme in a black 384 well microtiter plate. The fluorescence intensity is measured (excitation: 485 nm, emission: 555 nm) once every minute for 15 minutes in each well at 25° C. and the Vₘₐₓ is calculated as the slope of the plot of fluorescence intensity against time. The plot should be linear and the residual activity assay has been adjusted so that the diluted reference enzyme solution is within the linear range of the activity assay.

### Glucoamylase Activity Assay (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12M; 0.15M NaCl |
| pH: | 7.60 ± 0.05 |
| Incubation temperature: | 37° C. ± 1 |
| Reaction time: | 5 minutes |
| Wavelength: | 340 nm |

### Beta-Amylase Activity (BAMU)

Beta-amylase activity in BAMU is measured relative to a Novozymes beta-amylase standard. Beta-amylase acts on the non-reducing end of maltohexaose (G6) to form maltose (G2) and maltotetraose (G4). Produced G4 reacts stronger than G6 in the presence of lactose-oxidase and O2 to form H2O2. The formed H2O2 activates in the presence of peroxidase the oxidative condensation of 4-aminoantipyrine (AA) and N-ethyl-N-sulfopropyl-m-toluidine (TOPS), to form a purple product which can be quantified by its absorbance at 540 nm.

| Reaction conditions | |
|---|---|
| Buffer | 67 mM phosphate and |
| | 67 mM citrate |
| pH | 5.5 |
| Beta-amylase | 0.083-0.166 BAMU/mL |
| Maltohexaose | 0.856 mM |
| Lactose oxidase | 4.8 LOXU/mL |
| 4-Aminoantipyrine (AA) | 1.7 mM |
| N-ethyl-N-sulfopropyl-m- | 4.3 mM |
| toluidine (TOPS) | |
| Peroxidase (Sigma) | 2.1 U/mL |
| Temperature: | 37° C. |
| Reaction time: | 200 seconds |
| Wavelength | 540 nm |

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca2+; and pH 5.6) dextrinizes 5260 mg starch dry substance Merck Amylum solubile.

### Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, glucoamylase wildtype Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

Alpha-amylase

Starch+Iodine→Dextrins+Oligosaccharides
40° C., pH 2.5

Blue/violet t=23 sec. Decoloration

Standard Conditions/Reaction Conditions: (Per Minute)

Substrate: Starch, approx. 0.17 g/L

Buffer: Citate, approx. 0.03 M

Iodine (12): 0.03 g/L

CaCl2: 1.85 mM

pH: 2.50±0.05

Incubation temperature: 40° C.

Reaction time: 23 seconds

Wavelength: lambda=590 nm

Enzyme concentration: 0.025 AFAU/mL

Enzyme working range: 0.01-0.04 AFAU/mL

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 ml diluted sample or standard is incubated at 40° C. for 2 minutes. 0.5 ml 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### Determination of Sugar Profile and Solubilised Dry Solids

The sugar composition of the starch hydrolysates is determined by HPLC and glucose yield is subsequently calculated as DX. °BRIX, solubilized (soluble) dry solids of the starch hydrolysates are determined by refractive index measurement.

### Materials

### Alpha-Amylase Variants

The alpha-amylase variants tested are variants of LE399 (SEQ ID NO: 14, previously disclosed in, e.g., WO 2002/010355), as described in e.g., WO 2013/057143. LE399 comprises amino acids 1-37 of the alpha-amylase from Bacillus amyloliquefaciens (SEQ ID NO: 6) and amino acids 40-483 of the alpha-amylase from Bacillus licheniformis (SEQ ID NO: 1) with the following substitutions G48A T49I G107A H156Y A181T N190F I201F A209V Q264S. The substitutions in each variant as listed in the tables below are substitutions as compared to LE399. The position numbering is according to SEQ ID NO: 1.

LE399 is two amino acids shorter than SEQ ID NO: 1 in the N-terminal, i.e. there are no amino acids corresponding to positions 1 and 2 of SEQ ID NO: 1 in LE399. The alteration denoted in the tables as *2aH means insertion of H before the N-terminal V of LE399. A similar alteration in SEQ ID NO: 1 would be substitution of amino acid N2 with H, i.e. N2H (alternatively, deletion of amino acid A1 combined with substitution of amino acid N2 with H, i.e. A1* N2H). Likewise, the alterations denoted in the tables as *2aH *2bW means insertion of HW before the N-terminal V of LE399. A similar alteration in SEQ ID NO: 1 would be the substitutions A1H N2W.

Alpha-Amylase Variant A:

### H68W+K176L+E185P+I201Y+H205Y+K213T+Q360S+D416V+R437W

Alpha-Amylase Variant B:

### H142W+K176L+E185P+I201Y+H205Y+K213T+Q360S+D416V+R437W

Alpha-Amylase Variant C:

### N2H+H133Y+K176L+E185P+Q360S+R437W

Alpha-Amylase Variant D:

### K176L+E185P+I201Y+H205Y+K213T+Q360S+D416V+R437W

Alpha-Amylase Variant E:

### K176L+E185P+F201Y+1-1205Y+K213T+K315M+O360S+D416V+R437W

### Glucoamylase

Glucoamylase A: Glucoamylase from Trametes cingulata (WO 2006/069289).

Glucoamylase B: Glucoamylase from Aspergillus niger (Glucoamylase G1 derived from Aspergillus niger disclosed in Boel et al. (1984), EMBO J. 3 (5), 1097-1102, available from Novozymes A/S).

Glucoamylase C: Glucoamylase from Gloeophyllum trabeum disclosed in application no. EP13165995 as variant having the double substitution S95P, A121P.

### Beta-Amylase

Beta-Amylase A: Optimalt BBA (available from DuPont).

Beta-Amylase B: Beta-amylase from Bacillus flexus (U.S. Pat. No. 8,486,682).

### Comparative Amylase

Amylase A: Acid fungal alpha-amylase (AFAA) comprising the Rhizomucor pusillus alpha-amylase catalytic domain and the Aspergillus niger glucoamylase linker and CBM disclosed as V039 in Table 5 in WO 2006/069290.

Amylase B: Acid fungal alpha-amylase (AFAA) comprising the Rhizomucor pusillus alpha-amylase catalytic domain and the Aspergillus niger glucoamylase linker and CBM disclosed as variant PE96 in WO 2013/006756.

### Example 1

A slurry with 30% dry solids (DS) granular starch is prepared by adding common corn starch under stirring to water. The pH is adjusted with HCl to 4.5. The granular starch slurry is distributed to 100 ml blue cap flasks with 75 g in each flask. The flasks are incubated with magnetic stirring in a 60° C. water bath. At zero hours the 0.05 mg/g DS alpha-amylase variant and 0.1 mg/g DS glucoamylase are dosed to the flasks. Samples are withdrawn after 4, 24 and 48 hours, as indicated.

Total dry solids starch is determined using the following method. The starch is completely hydrolyzed by adding an excess amount of alpha-amylase (300 KNU/Kg dry solids) and subsequently placing the sample in an oil bath at 95° C. for 45 minutes. After filtration through a 0.22 microM filter the dry solids is measured by refractive index measurement.

Soluble dry solids in the starch hydrolysate are determined on samples after filtering through a 0.22 microM filter. Soluble dry solids are determined by refractive index measurement and the sugar profile was determined by HPLC. The amount of glucose is calculated as % DX.

### Example 2

Powdered corn starch was combined in a plastic flask with tap water to produce a 31.5% solids starch slurry. The slurry was adjusted to a pH of 4.5 using a 0.1M HCl solution. 20 mL aliquots of this slurry were added to 30 mL Nalgene screw top plastic bottles and the weights were recorded. Each of the enzyme-treated bottles was given an alpha amylase dosage of 0.05 mg enzyme protein per gram of starch dry substance (mg EP/g DS), and a glucoamylase dose of 0.1 mg EP/g DS. Two bottles were left un-dosed as controls. Once dosed, the bottles were placed into two rotisserie ovens which were set to 60° C. or 66° C. respectively.

At various time points (T=4, 24, and 48 hours), 2 mL aliquots were removed from each flask and placed into pre-weighed 15 mL conical spin tubes which were then re-weighed. Each sample was deactivated with 18 μL of 1M HCl. After deactivation, the samples were vortexed completely. The supernatant in each tube was filtered through a 0.45 μm filter disc into microcentrifuge tubes for HPLC analysis. These microcentrifuge tubes were then placed in boiling water for 10 minutes to completely deactivate any enzyme activity. HPLC samples were prepared in vials by diluting 50 μL of the filtered sample in each Eppendorf tube with 950 μL of 0.005M H2SO4 to yield a 20:1 dilution factor.

Once the supernatant had been removed from each sample, the tubes were filled to the 10 mL mark with tap water. The tubes were then vortexed and centrifuged as above, then decanted. This process was repeated a second time with tap water, followed by a third and final time with an 80% ethanol solution to remove residual solubles from each tube's solids. These tubes were placed into a 55° C. oven for a minimum of 24 hours before being placed in a 105° C. oven for 24 hours to dry completely. Immediately after being taken out of the oven, the tube caps were closed to prevent the tube contents from picking up moisture. The tubes were then weighed on an analytical balance to determine the weight of the contents.

### Determination of Solubilisation by FIS Analysis

The fraction of solids which had been solublized was calculated as follows:

$$\% Solubilisation = 1 - \frac{\% Insoluble Solids In Enzyme Treated Tube @ {T}_{X}}{\% Insuluble Solids In Control Tube @ {T}_{x}}$$

### HPLC Analysis

HPLC system Agilent's 1100/1200 series with Chem station software
   Degasser, Quaternary Pump, Auto-Sampler, Column Compartment w/ Heater Refractive Index Detector (RI)
Column Bio-Rad HPX-87H Ion Exclusion Column, 300 mm×7.8 mm, part #125-0140
   Bio-Rad guard cartridge cation H, part #125-0129, Holder part #125-0131
Method 0.005M H2SO4 mobile phase
   Flow rate: 0.6 ml/min
   Column temperature: 65° C.
   RI detector temperature: 55° C.

The method quantified analytes using calibration standards for DP4+, DP3, DP2, glucose, fructose, acetic acid, lactic acid, glycerol and ethanol (% w/v). A four point calibration including the origin was used for quantification.

For this experiment, organic acids and fructose HPLC results were not compared among treatments.

TABLE 1
Soluble dry solids as percentage of total dry substance.

| | Glucoamylase A | | | Glucoamylase A (at | | | Glucoamylase C | | |
|---|---|---|---|---|---|---|---|---|---|
| Alpha-amylase | (at 60° C.) | | | 66° C.) | | | (at 60° C.) | | |
| Variant | 4 hr | 24 hr | 48 hr | 4 hr | 24 hr | 48 hr | 4 hr | 24 hr | 48 hr |
| Alpha-Amylase Variant A | 56.8% | 88.0% | 91.9% | 56.2% | 85.6% | 90.2% | 57.6% | 85.0% | 88.5% |
| Alpha-Amylase Variant B | 48.2% | 81.1% | 85.8% | 49.3% | 79.3% | 83.3% | 49.3% | 81.6% | 85.3% |
| Alpha-Amylase Variant C | n.d. | n.d. | n.d. | n.d. | n.d. | n.d. | 53.7% | 83.1% | 85.0% |
| Alpha-Amylase Variant D | n.d. | 71.8% | 74.6% | 55.5% | 83.9% | 86.5% | n.d. | 70.5% | 73.9% |

*n.d. = not determined

TABLE 2
DX of the soluble hydrolysate.

| | Glucoamylase A | | | Glucoamylase A (at | | | Glucoamylase C | | |
|---|---|---|---|---|---|---|---|---|---|
| Alpha-amylase | (at 60° C.) | | | 66° C.) | | | (at 60° C.) | | |
| Variant | 4 hr | 24 hr | 48 hr | 4 hr | 24 hr | 48 hr | 4 hr | 24 hr | 48 hr |
| Alpha-Amylase Variant A | 84.8% | 95.1% | 95.5% | 81.5% | 92.8% | 95.2% | 88.4% | 96.1% | 95.4% |
| Alpha-Amylase Variant B | 88.9% | 94.3% | 95.6% | 83.3% | 90.6% | 94.1% | 92.9% | 95.9% | 95.1% |
| Alpha-Amylase Variant C | n.d. | n.d. | n.d. | n.d. | n.d. | n.d. | 90.5% | 95.9% | 95.2% |
| Alpha-Amylase Variant D | n.d. | 93.9% | 93.8% | 81.3% | 92.9% | 95.2% | n.d. | 95.1% | 93.2% |

*n.d. = not determined

This example demonstrates that the tested alpha-amylase variants in combination with glucoamylase effectively solubilize starch (more than 70% in 48 hours) and give a rather high % DX (>95%).

### Example 3

This comparative example illustrates the lower starch solubilisation of acid fungal alpha-amylase known to have activity on raw starch, also combined with glucoamylase, following the protocol of Example 1 at 60° C. Samples were collected at 4 hours, 24 hours and either 48 or 98 hours. The results are shown in tables 3 and 4.

TABLE 3
Soluble dry solids as percentage of total dry substance.

| Comparative | | | | | | | | |
|---|---|---|---|---|---|---|---|---|
| alpha- | Glucoamylase A | | | | Glucoamylase B | | | |
| amylase | 4 hr | 24 hr | 48 hr | 98 hr | 4 hr | 24 hr | 48 hr | 98 hr |
| Amylase A | 33.8% | 55.8% | n.d. | 63.9% | 21.7% | 46.5% | n.d. | 56.3% |
| Amylase B | 38.6% | 65.6% | 72.9% | n.d. | 25.4% | 54.3% | n.d. | 64.9% |

n.d. = not determined

TABLE 4
DX of the soluble hydrolysate.

| Comparative | | | | | | | | |
|---|---|---|---|---|---|---|---|---|
| alpha- | Glucoamylase A | | | | Glucoamylase B | | | |
| amylase | 4 hr | 24 hr | 48 hr | 98 hr | 4 hr | 24 hr | 48 hr | 98 hr |
| Amylase A | 94.7% | 95.9% | n.d. | 94.8% | 82.5% | 95.5% | n.d. | 95.8% |
| Amylase B | 93.7% | 95.4% | 94.5% | n.d. | 79.0% | 94.7% | n.d. | 95.1% |

n.d. = not determined

This example demonstrates that the acid fungal alpha-amylase Amylase A and Amylase B, which is known to have activity on raw starch, but which is not a low pH bacterial amylase, in combination with glucoamylase, showed lower starch solubilisation than the tested alpha-amylase variants of Example 1, which are low pH bacterial amylases, in combination with glucoamylase.

### Example 4

Powdered corn starch was combined in a plastic flask with DI water to produce a 40.5% solids starch slurry. The slurry was adjusted to a pH of 4.5 using a 0.1M HCl solution. 10 mL aliquots of this slurry were added to 30 mL Nalgene screw top plastic bottles and the weights were recorded. Each of the bottles dosed according to their weights with alpha amylase and glucoamylase enzymes according to Table 5. Sufficient water was added to each bottle to reach 30% dry solids. Bottles 1 and 2 were left undosed and were treated as blanks. Once dosed, the bottles were placed into two rotisserie ovens which were set to 60° C. or 66° C. according to Table 5. The blanks were placed in 60° C. to avoid starch gelatinization.

TABLE 5
Dosing table of alpha-amylase (AA) and glucoamylase (AMG).

| | Alpha- | | | | | | | |
|---|---|---|---|---|---|---|---|---|
| | Amylase | | AA | AMG | Temp | | DS | total |
| Bottles | (AA) | AMG | dose | Dose | (° C.) | pH | (%) | dose |
| 1, 2 | Blank (30% solids) | | | | 60 | 4.5 | 30 | 0 |
| 3, 4 | Alpha- | Gluco- | 0.045 | 0.09 | 66 | 4.5 | 30 | 0.135 |
| | Amylase | amylase | | | | | | |
| | Variant A | C | | | | | | |
| 5, 6 | Alpha- | Gluco- | 0.05 | 0.1 | 66 | 4.5 | 30 | 0.15 |
| | Amylase | amylase | | | | | | |
| | Variant A | C | | | | | | |
| 7, 8 | Alpha- | Gluco- | 0.045 | 0.09 | 66 | 4.5 | 30 | 0.135 |
| | Amylase | amylase | | | | | | |
| | Variant E | C | | | | | | |
| 9, 10 | Alpha- | Gluco- | 0.05 | 0.1 | 66 | 4.5 | 30 | 0.15 |
| | Amylase | amylase | | | | | | |
| | Variant E | C | | | | | | |
| 11, 12 | Alpha- | Gluco- | 0.1 | 0.2 | 66 | 4.5 | 30 | 0.3 |
| | Amylase | amylase | | | | | | |
| | Variant E | C | | | | | | |
| 13, 14 | Alpha- | Gluco- | 0.2 | 0.4 | 66 | 4.5 | 30 | 0.6 |
| | Amylase | amylase | | | | | | |
| | Variant E | C | | | | | | |
| 15, 16 | Alpha- | Gluco- | 0.1 | 0.1 | 66 | 4.5 | 30 | 0.2 |
| | Amylase | amylase | | | | | | |
| | Variant E | C | | | | | | |

At 24 and 48 hour time points, 2 mL aliquots were removed from each flask and placed into pre-weighed 15 mL conical spin tubes which were then re-weighed. Each sample was deactivated with 18 μL of 1M HCL. After deactivation, the samples were vortexed and put in centrifuge for 10 minutes at 3500 rpm. The supernatant in each tube was filtered through a 0.45 μm filter disc into small microcentrifuge tubes for HPLC analysis of hydrolyzate. These microcentrifuge tubes were then placed in boiling water for 10 minutes to completely deactivate any enzyme activity. HPLC samples were prepared in vials by diluting 50 μL of the filtered sample in with 950 μL of mobile phase (5 mM H₂SO₄) to yield a 20:1 dilution factor. HPLC samples were analyzed according to procedure described in Example 2. Starch solubilization was also determined according to the procedure described in Example 2.

TABLE 6
Soluble dry solids as percentage of total dry substance
(average of replicates).

| | | Alpha-Amylase | | Alpha-Amylase | |
|---|---|---|---|---|---|
| Treatments | | Variant A/ | | Variant E/ | |
| AA dose | AMG dose | Glucoamylase C | | Glucoamylase C | |
| (mg/gDS) | (mg/gDS) | 24 hr | 48 hr | 24 hr | 48 hr |
| 0.045 | 0.09 | 73.2% | 78.0% | 78.4% | 82.5% |
| 0.05 | 0.1 | 76.8% | 80.0% | 80.7% | 84.3% |
| 0.1 | 0.2 | n.d. | n.d. | 89.2% | 92.5% |
| 0.2 | 0.4 | n.d. | n.d. | 93.4% | 95.7% |

*n.d. = not determined

TABLE 7
DX of the soluble hydrolyzate (average of replicates).

| | | Alpha-Amylase | | Alpha-Amylase | |
|---|---|---|---|---|---|
| Treatments | | Variant A/ | | Variant E/ | |
| AA dose | AMG dose | Glucoamylase C | | Glucoamylase C | |
| (mg/gDS) | (mg/gDS) | 24 hr | 48 hr | 24 hr | 48 hr |
| 0.045 | 0.09 | 97.4% | 96.5% | 97.2% | 96.3% |
| 0.05 | 0.1 | 97.2% | 96.2% | 97.2% | 96.1% |
| 0.1 | 0.2 | n.d. | n.d. | 96.1% | 94.1% |
| 0.2 | 0.4 | n.d. | n.d. | 94.1% | 90.9% |

*n.d. = not determined

This example demonstrates that the tested alpha-amylase variants in combination with glucoamylase effectively solubilize starch (more than 70% in 48 hours) and give a rather high % DX (>95%).

### Example 5

Powdered corn starch was combined in a plastic flask with DI water to produce a 40.5% solids starch slurry. The slurry was adjusted to a pH of 4.5 using a 0.1M HCl solution. 10 mL aliquots of this slurry were added to 30 mL Nalgene screw top plastic bottles and the weights were recorded. Each of the bottles dosed according to their weights with alpha amylase and beta amylase enzymes according to Table 8. Sufficient water was added to each bottle to reach 30% dry solids. Bottles 21 and 22 were left undosed and were treated as blanks. Once dosed, the bottles were placed into two rotisserie ovens which were set to 60° C. or 66° C. according to Table 8. The blanks were placed in 60° C. to avoid starch gelatinization.

TABLE 8
Dosing table of alpha-amylase and beta-amylase.

| | Alpha- | Beta- | | | | | | |
|---|---|---|---|---|---|---|---|---|
| | Amylase | Amylase | AA dose | BA Dose | Temp | | DS | total |
| Bottles | (AA) | (BA) | (mg/gDS) | (mg/gDS) | (° C.) | pH | (%) | dose |
| 1, 2 | Alpha- | Beta- | 0.05 | 0.1 | 66 | 4.5 | 30 | 0.15 |
| | Amylase | Amylase | | | | | | |
| | Variant A | B | | | | | | |
| 3, 4 | Alpha- | Beta- | 0.05 | 0.15 | 66 | 4.5 | 30 | 0.2 |
| | Amylase | Amylase | | | | | | |
| | Variant A | B | | | | | | |
| 5, 6 | Alpha- | Beta- | 0.05 | 0.15 | 66 | 4.5 | 30 | 0.2 |
| | Amylase | Amylase | | | | | | |
| | Variant E | A | | | | | | |
| 7, 8 | Alpha- | Beta- | 0.05 | 0.1 | 66 | 4.5 | 30 | 0.15 |
| | Amylase | Amylase | | | | | | |
| | Variant A | A | | | | | | |
| 9, 10 | Alpha- | Beta- | 0.05 | 0.15 | 66 | 4.5 | 30 | 0.2 |
| | Amylase | Amylase | | | | | | |
| | Variant A | A | | | | | | |
| 11, 12 | Alpha- | Beta- | 0.05 | 0.15 | 66 | 4.5 | 30 | 0.2 |
| | Amylase | Amylase | | | | | | |
| | Variant E | A | | | | | | |
| 13, 14 | Alpha- | Beta- | 0.05 | 0.15 | 60 | 4.5 | 30 | 0.2 |
| | Amylase | Amylase | | | | | | |
| | Variant A | B | | | | | | |
| 15, 16 | Alpha- | Beta- | 0.05 | 0.15 | 60 | 4.5 | 30 | 0.2 |
| | Amylase | Amylase | | | | | | |
| | Variant A | A | | | | | | |
| 17, 18 | Alpha- | Beta- | 0.05 | 0.15 | 60 | 4.5 | 30 | 0.2 |
| | Amylase | Amylase | | | | | | |
| | Variant E | B | | | | | | |
| 19, 20 | Alpha- | Beta- | 0.05 | 0.15 | 60 | 4.5 | 30 | 0.2 |
| | Amylase | Amylase | | | | | | |
| | Variant E | A | | | | | | |

| | | | | | | | |
|---|---|---|---|---|---|---|---|
| 21, 22 | Blank (30% solids) | 0.1 | 0.1 | 60 | 4.5 | 30 | 0.2 |

At 24 and 48 hour time points, 2 mL aliquots were removed from each flask and placed into pre-weighed 15 mL conical spin tubes which were then re-weighed. Each sample was deactivated with 18 μL of 1M HCL. After deactivation, the samples were vortexed and put in centrifuge for 10 minutes at 3500 rpm. The supernatant in each tube was filtered through a 0.45 μm filter disc into small microcentrifuge tubes for HPLC analysis of hydrolyzate. These microcentrifuge tubes were then placed in boiling water for 10 minutes to completely deactivate any enzyme activity. HPLC samples were prepared in vials by diluting 50 μL of the filtered sample in with 950 μL of mobile phase (5 mM H₂SO₄) to yield a 20:1 dilution factor. HPLC samples were analyzed according to procedure described in Example 2. Starch solubilization was also determined according to the procedure described in Example 2.

TABLE 9
Soluble dry solids as percentage of total dry substance (average of replicates).

| | | | Alpha-Amylase Variant A | | | | Alpha-Amylase Variant E | | | |
|---|---|---|---|---|---|---|---|---|---|---|
| Treatments | | | Beta-Amylase | | Beta-Amylase | | Beta- | | Beta-Amylase | |
| AA dose | BA dose | Temperature | A | | B | | Amylase A | | B | |
| (mg/gDS) | (mg/gDS) | (° C.) | 24 hr | 48 hr | 24 hr | 48 hr | 24 hr | 48 hr | 24 hr | 48 hr |
| 0.05 | 0.1 | 66° C. | 44.7% | 53.0% | 61.7% | 65.5% | n.d. | n.d. | n.d. | n.d. |
| 0.05 | 0.15 | | 46.3% | 53.8% | 59.9% | 66.2% | 52.1% | 59.2% | 65.7% | 71.4% |
| 0.05 | 0.15 | 60° C. | 32.4% | 38.7% | 51.3% | 58.0% | 35.2% | 42.7% | 55.9% | 62.4% |

TABLE 10
% DP2 of soluble hydrolyzate (average of replicates).

| | | | Alpha-Amylase Variant A | | | | Alpha-Amylase Variant E | | | |
|---|---|---|---|---|---|---|---|---|---|---|
| Treatments | | | Beta-Amylase | | Beta-Amylase | | Beta-Amylase | | Beta-Amylase | |
| AA dose | BA dose | Temperature | A | | B | | A | | B | |
| (mg/gDS) | (mg/gDS) | (° C.) | 24 hr | 48 hr | 24 hr | 48 hr | 24 hr | 48 hr | 24 hr | 48 hr |
| 0.05 | 0.1 | 66° C. | 19.1% | 20.6% | 42.6% | 40.6% | n.d. | n.d. | n.d. | n.d. |
| 0.05 | 0.15 | | 20.1% | 21.4% | 44.3% | 40.9% | 19.8% | 21.1% | 44.1% | 42.0% |
| 0.05 | 0.15 | 60° C. | 22.3% | 22.9% | 57.0% | 56.2% | 21.7% | 22.1% | 56.3% | 55.5% |

This example demonstrates that the tested alpha-amylase variants in combination with beta-amylase effectively solubilize starch and can work effectively even at higher temperature of 66° C.

1. A process for increasing starch solubilisation comprising:
a) subjecting an aqueous granular starch slurry to an alpha-amylase variant comprising an alteration at one or more positions corresponding to any of positions 1, 2, 68, 71, 126, 133, 142, 144, 156, 158, 176, 185, 201, 205, 213, 239, 279, 316, 318, 360, 416, 437 and 450 of SEQ ID NO: 1 at a temperature below or just above the initial gelatinization temperature of said granular starch;
b) in the presence of a carbohydrate-source generating enzyme;
thereby increasing solubilisation of the granular starch.

2. The process of claim 1, wherein the process is performed at a temperature of about 60-70° C.

3. The process of claim 1, wherein the alpha-amylase variant is added in an amount greater than 0.05 mg enzyme protein per gram dry solids.

4. The process of claim 1, wherein the alpha-amylase variant comprises a substitution at position 176 and/or 185 of SEQ ID NO: 1.

5. The process of claim 1, wherein the alpha-amylase variant comprises a substitution at one or more of positions 176, 185, 360 and/or 437 of SEQ ID NO: 1.

6. The process of claim 1, comprising a glucoamylase and a beta-amylase.

7. The process of claim 1, further comprising addition of an enzyme selected from the group consisting of cyclodextrin glucanotransferase, fungal alpha-amylase, Bacillus alpha-amylase, barley beta-amylase, isoamylase, pullulanase, or a combination thereof.

8. The process of claim 1, wherein the starch slurry has 20-55% dry solids granular starch.

9. The process of claim 1, wherein at least 85%, of the dry solids of the granular starch is converted into a soluble starch hydrolysate.

10. The process of claim 1, wherein the temperature is at least 58° C.

11. The process of claim 1, wherein the pH is in the range of 3.0 to 7.0.

12. The process of claim 1, wherein the soluble starch hydrolysate has a DX of at least 94.5%.

13. The process of claim 1, wherein the dominating saccharide in the soluble starch hydrolysate is glucose or maltose.

14. The process of claim 1, wherein the granular starch is obtained from tubers, roots, stems, or whole grain.

15. The process of claim 1, wherein the granular starch is obtained from cereals.

16. The process of claim 1, wherein the granular starch is obtained from corn, cobs, wheat, barley, rye, milo, sago, cassava, tapioca, sorghum, rice or potatoes.

17. The process of claim 1, wherein the granular starch is obtained from dry milling of whole grain or from wet milling of whole grain.

18. The process of claim 1, wherein the process is conducted in an ultrafiltration system and where the retentate is held under recirculation in presence of enzymes, raw starch and water and where the permeate is the soluble starch hydrolysate.

19. A process for production of high fructose starch-based syrup (HFSS), wherein a soluble starch hydrolysate of the process of claim 1 is subjected to conversion into high fructose starch-based syrup (HFSS).

20. A process for production of maltose syrup, wherein a soluble starch hydrolysate of the process of claim 1 is subjected to conversion into maltose syrup.
